(19) United States
(12) Patent Application Publication
(10) Pub. No.: US 2020/0080983 A1
(43) Pub. Date: Mar. 12, 2020

(54) A TOXICANT MONITORING SYSTEM

(71) Applicant: National University of Singapore, Singapore (SG)

(72) Inventors: How Yong NG, Singapore (SG); Shailesh KHARKWAL, Singapore (SG); Yi Chao TAN, Singapore (SG)

(21) Appl. No.: 16/312,809

(22) PCT Filed: Jun. 29, 2017
(86) PCT No.: PCT/SG2017/050323

§ 371 Date: Dec. 21, 2018

(30) Foreign Application Priority Data
Jun. 29, 2016 (SG) 10201605369Y

(51) Int. Cl. G01N 33/18; G01N 27/416; H01M 8/16

(52) U.S. Cl. CPC G01N 33/1866; G01N 27/4167; G01N 2033/184; G01N 27/4168; H01M 8/16; G01N 33/1826

(57) ABSTRACT

There is provided a toxicant monitoring system for continuously monitoring level of a toxicant in wastewater comprising: a microbial electrochemical sensor; an electrical sensor; a process controller configured to execute instructions for monitoring the level of toxicant in the wastewater, the instructions including collection of a sample by an auto-sampler and generation of a notification by a telecommunication system; an input port to provide wastewater and fuel to the microbial electrochemical sensor; and an output port to receive the wastewater after the wastewater contacts the microbial electrochemical sensor.

### TECHNICAL FIELD

The present invention relates to a toxicant monitoring system for continuously monitoring level of a toxicant in wastewater.

### BACKGROUND

Industrial effluents can often be heavily loaded with complex inorganic and organic chemicals including toxic substances such as pesticides, pharmaceuticals, dyes, petro-chemicals, detergents, surfactants, heavy metals, cyanide and the like. If these toxic substances are discharged into a water source, the water cannot be safely used as drinking water. Further, release of these toxic substances as a concentrated stream into the main sewer network may affect activity and viability of microorganisms in biological treatment processes such as the activated sludge, thereby impeding the performance of the activated sludge process in wastewater treatment plants. If this occurs, the treated wastewater would contain higher organic and suspended solid concentrations, violating the discharge limits and would potentially also affect the process of water treatment downstream of the water bodies into which the treated wastewater is discharged.

Influent wastewater is not routinely screened for toxicants in treatment plants. Even if the influent wastewater is screened, the assessment methods are offline. These assessment methods include bioassays and chemical analysis of the wastewater. However, offline methods usually need auxiliary procedures and are time consuming. Online methods, which utilise online biosensors comprising DNA, enzymes, engineered bacteria or activated sludge give a fast response but their operation is usually complex and costly.

An example of a method of online toxicant monitoring which is known utilises a microbial fuel cell (MFC). In particular, the anode of a MFC is usually covered by a biofilm comprising electrochemically active bacteria. Organic matter comprised in the wastewater is metabolized in the anode chamber by the electrochemically active bacteria, thereby producing electrons which pass through an external circuit to the cathode where reduction takes place, and current or voltage is produced which is proportional to the utilization rate of the organic matter. When the biofilm is subjected to toxicants, the normal electron transport metabolism of the electrochemically active bacteria is inhibited, causing current/voltage to decrease. Through monitoring the changes in current/voltage, the MFC is capable of on-line and real-time sensing of toxicants in the water. However, the utilisation of MFC as a toxicant sensor in a practical application has not been reported. It would therefore be obvious to a person skilled in the art that there would be many challenges in using a MFC in a practical application due to the complexity and dynamic fluctuation of wastewater qualities.

There is therefore a need for an improved online toxicant monitoring system.

### SUMMARY OF THE INVENTION

The present invention seeks to address these problems, and/or to provide an improved toxicant monitoring system.

In general terms, the invention relates to a comprehensive toxicant monitoring system which allows for continuous and real-time monitoring of the level of a toxicant in water. In particular, the toxicant monitoring system of the present invention allows for continuous detection and measurement of toxic compounds in various water sources including those which are low in organic strength. Even more in particular, the toxicant monitoring system of the present invention allows the detection of toxicants, immediate collection of a sample of the toxic water, data logging and transmission of a notification to a user of the system via a telecommunication system.

The advantage of the toxicant monitoring system of the present invention is that the system requires minimum maintenance and may be easily integrated into existing wastewater systems, for example by employing it upstream of an activated sludge process in a wastewater treatment plant, at pumping stations of the wastewater or at the wastewater discharge points. The toxicant monitoring system may also be integrated in a river water network by employing it at strategic locations along the river or at the raw water intake point. In other words, the system of the present invention may serve as a sustainable source control method for online and continuous monitoring of toxicants in water including wastewater.

According to a first aspect, the present invention provides a toxicant monitoring system for continuously monitoring level of a toxicant in wastewater comprising:
a microbial electrochemical sensor;
an electrical sensor electrically coupled to the microbial electrochemical sensor, the electrical sensor configured to measure at least one measurement of voltage, current, power, or hydrogen produced, wherein a drop in the measurement correlates to the presence of a toxicant;
a process controller in communication with the electrical sensor, the process controller configured to execute instructions for monitoring the level of the toxicant in the wastewater, wherein the process controller is in further communication with an auto-sampler and a telecommunication system, the auto-sampler configured to collect a sample from the wastewater when triggered, and the telecommunication system configured to generate a notification when triggered;
an input port configured to provide a fuel and wastewater to the microbial electrochemical sensor; and an output port configured to receive the wastewater after the wastewater contacts the microbial electrochemical sensor.

For the purposes of the present invention, toxicant is defined as including any substance or compound that is harmful to the environment or biological health. Examples of toxicants include, but are not limited to, heavy metals, cyanide, nitrates, sulphates, and extreme fluctuations of pH.

For the purposes of the present invention, wastewater is defined as comprising any water source including, but not limited to, effluent, river water, lake water and groundwater. The effluent may be from any source such as, but not limited to, industrial effluent, electroplating waste, mining waste, silver plating waste, metallurgy waste, textile manufacturing waste, leather processing waste, or pesticide manufacturing waste.

The microbial electrochemical sensor may be any suitable microbial electrochemical sensor for the purposes of the present invention. In particular, the microbial electrochemical sensor may comprise an anode and a cathode, wherein the anode comprises microbes. The cathode may comprise either catalysts or microbes. For example, the catalysts may include, but are not limited to, platinum, manganese, titanium, cobalt, indium, tungsten and a combination thereof.

The microbes on the anode and cathode may be in the form of a biofilm on the anode. In the microbial electrochemical sensor, the anode and the cathode may be separated by an ion exchange membrane, such as a proton exchange membrane. In particular, the microbial electrochemical sensor may be a microbial fuel cell or a microbial electrolysis cell. Even more in particular, the microbial electrochemical sensor may be a microbial electrolysis cell.

According to a particular aspect, the microbial electrochemical sensor may comprise one or more microbial electrochemical sensors. In particular, the toxicant monitoring system may comprise at least two microbial electrochemical sensors. Even more in particular, the toxicant monitoring system comprises at least three microbial electrochemical sensors.

According to a particular aspect, the fuel provided to the microbial electrochemical sensor may be a source of organics. The source of organics may comprise any suitable source such as, but not limited to, sodium acetate, glucose, sucrose, xylose, starch, cellulose, synthetic wastewater, domestic wastewater, food processing wastewater, and lignocellulosic biomass.

The fuel provided to the microbial electrochemical sensor may be for forming and regenerating the microbial film on the anode. The fuel may also be for maintaining and/or balancing the concentration of organics in the wastewater when the wastewater is of low organic strength.

According to a particular aspect, the toxicant monitoring system may further comprise at least one of the following:
a first reservoir fluidly coupled to the input port, the first reservoir comprising the fuel;
a second reservoir fluidly coupled to the input port, the second reservoir comprising the wastewater;
a third reservoir fluidly coupled to the output port, the third reservoir comprising the wastewater which has contacted the microbial electrochemical sensor;
a first flow control device fluidly coupled to the input port, the first flow control device configured to adjust flow of the fuel from the first reservoir to the input port;
a second flow control device fluidly coupled to the input port, the second flow control device configured to adjust a flow of the wastewater from the second reservoir to the input port; and
a level sensor fluidly coupled to the second reservoir and in communication with the process controller, the level sensor configured to send a signal to the process controller when there is no wastewater in the second reservoir.

According to a particular aspect, the third reservoir may be fluidly coupled to the second reservoir via a third flow control device, the third flow control device configured to adjust a flow of the wastewater from the third reservoir to the second reservoir.

The process controller may also be in communication with at least one of the first flow control device, the second flow control device and the third flow control device.

### BRIEF DESCRIPTION OF THE DRAWINGS

In order that the invention may be fully understood and readily put into practical effect there shall now be described by way of non-limitative example only exemplary embodiments, the description being with reference to the accompanying illustrative drawings. In the drawings:

FIG. 1 shows a schematic diagram of the toxicant monitoring system according to an embodiment of the present invention;

FIG. 2 shows a schematic diagram of the toxicant monitoring system according to an embodiment of the present invention;

FIG. 3 shows a schematic diagram of the toxicant monitoring system according to an embodiment of the present invention;

FIG. 4 shows a schematic diagram of a microbial fuel cell (MFC) according to an embodiment of the present invention;

FIG. 5 shows the differences between a microbial electrolysis cell (MEC) and a MFC;

FIG. 6 shows the response of the MEC against Cu(II) when the anode potential is controlled at −0.1 V vs. Ag/AgCl (3M KCl) reference electrode;

FIG. 7 shows the conversion of a microbial electrochemical sensor from a MFC to a MEC;

FIG. 8 shows the algorithm of the data analysis of the toxicant monitoring system according to an embodiment of the present invention;

FIG. 9(a)-(e) shows the response of current with time (up to 120 minutes) in the presence of heavy metals: (a) Cu(II), (b) Cd(II), (c) Ni(II), (d) Zn(II) and (e) cyanide;

FIG. 10 shows the response of current with time during 0-10 h and 10-24 h with an acidic toxic event of pH varying between 3 and 6; and

FIG. 11 shows the response of current with time in the presence of Cu(II) in wastewater of high and low organic strength.

### DETAILED DESCRIPTION

As explained above, there is a need for improved toxicant monitoring system which allows for continuous monitoring of toxicant levels in a water source.

The present invention provides a real-time water quality monitoring system which enables monitoring and detection of toxicants, as well as collection of samples for further analysis. In the event of detecting a toxicant, the system also enables providing a warning to a user of the system. The toxicant monitoring system of the present invention may be used in conjunction with any suitable water source, including water sources with low organic strength. The system of the present invention provides a quick, preventative, easy-to-perform, inexpensive and online method for monitoring toxicant levels in a water source. Further, the system of the present invention may be amenable to operating on site and may be adapted as a mobile unit for in-situ sensing.

In general terms, the toxicant monitoring system of the present invention provides an online biomonitoring system comprising: microbial electrochemical sensor which comprise microbes in the form of a biofilm on an anode of the sensor, the microbes being sensitive to toxicants; monitoring and recording means which monitor and record the current, voltage, power or hydrogen produced; a signal processor communicating with the monitoring and recording means, wherein a reduction in the current, voltage, power or hydrogen produced correlates to the presence of toxicants; a process controller which applies the data collected by the signal processor to monitor the effect of diverting or controlling addition of a toxic influent, for example to activated sludge in a wastewater treatment system; and an organic food source to provide a baseline for the production of current, voltage, power or hydrogen for a water sample which is low in organic concentration. The system may also comprise a feed recirculation arrangement for a situation when there is no incoming water for testing.

According to a first aspect, there is provided a toxicant monitoring system for continuously monitoring level of a toxicant in wastewater comprising:
a microbial electrochemical sensor;
an electrical sensor electrically coupled to the microbial electrochemical sensor, the electrical sensor configured to measure at least one measurement of voltage, current, power, or hydrogen produced, wherein a drop in the measurement correlates to the presence of a toxicant;
a process controller in communication with the electrical sensor, the process controller configured to execute instructions for monitoring the level of the toxicant in the wastewater, wherein the process controller is in further communication with an auto-sampler and a telecommunication system, the auto-sampler configured to collect a sample from the wastewater when triggered, and the telecommunication system configured to generate a notification when triggered;
an input port configured to provide a fuel and wastewater to the microbial electrochemical sensor; and
an output port configured to receive the wastewater after the wastewater contacts the microbial electrochemical sensor.

For the purposes of the present invention, toxicant is defined as including any substance or compound that is harmful to the environment or biological health. Examples of toxicants include, but are not limited to, heavy metals, cyanide, nitrates, sulphates, and extreme fluctuations of pH. For example, the heavy metals may be cadmium, copper, zinc, nickel, and the like.

For the purposes of the present invention, wastewater is defined as comprising any water source including, but not limited to, effluent, river water, lake water and groundwater. The effluent may be from any source such as, but not limited to, industrial effluent, electroplating waste, mining waste, silver plating waste, metallurgy waste, textile manufacturing waste, leather processing waste, or pesticide manufacturing waste.

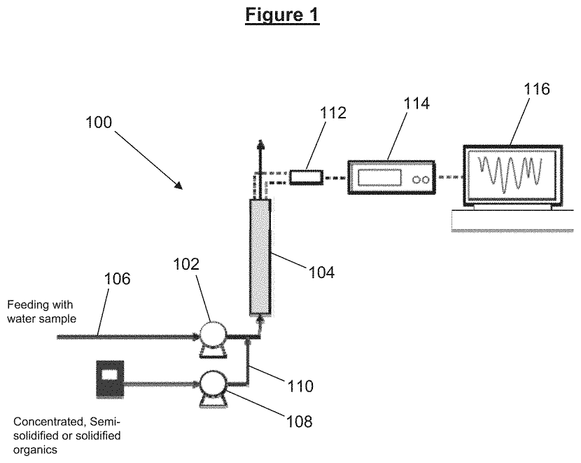

A simplified setup of the toxicant monitoring system 100 according to the present invention is shown in FIG. 1. In particular, a pump 102 feeds water into the microbial fuel cell or microbial electrolysis cell 104 by way of feed line 106. Optionally, pump 108 may supply organic food source, comprising concentrated, semi-solidified or solidified organic material into the microbial fuel cell or microbial electrolysis cell 104 by way of feed line 110. A resistor or potentiostat 112 controls the voltage difference between a cathode and an anode comprised in the microbial fuel cell or microbial electrolysis cell 104. There is also provided a multimeter 114 which measures the current, voltage, power, or hydrogen produced by the microbial fuel cell or microbial electrolysis cell 104. The information from the multimeter 114 is then fed into a process controller 116 to carry out data logging and apply appropriate algorithms to enable: detection and quantification of toxicants in the water fed into the microbial fuel cell or microbial electrolysis cell 104; immediate collection of a sample of the water when necessary; and transmission of a notification to a user of the system via a telecommunication system when necessary.

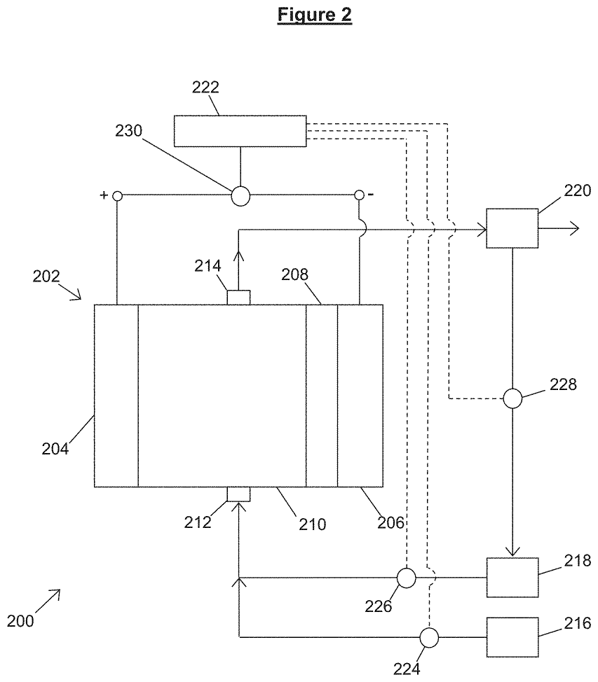

A toxicant monitoring system 200 for continuously monitoring level of a toxicant in wastewater according to one embodiment of the present invention is provided in FIG. 2.

System 200 comprises a microbial electrochemical sensor 202. The microbial electrochemical sensor 202 comprises anode 204, cathode 206, and ion-exchange membrane 208. In particular, the ion-exchange membrane 208 may be a proton-exchange membrane. A chamber 210 may be disposed between the ion-exchange membrane 208 and the anode 204, and may be configured to receive at least one fuel and wastewater. The fuel and wastewater may contact the anode 204 and the ion-exchange membrane 208.

In particular, the microbial electrochemical sensor 202 may comprise a microbial fuel cell or a microbial electrolysis cell. According to a particular embodiment, the microbial electrochemical sensor 202 may be a microbial electrolysis cell.

According to a particular embodiment, the microbial electrochemical sensor 202 may comprise one or more microbial fuel cell and/or microbial electrolysis cell. In particular, the microbial electrochemical sensor 202 may comprise at least two, three, four or more microbial fuel cells and/or microbial electrolysis cells. Even more in particular, the microbial electrochemical sensor 202 may comprise three microbial fuel cells and/or microbial electrolysis cells.

The microbial electrochemical sensor 202 may also comprise input port 212 configured to supply a fuel and wastewater to the chamber 210. The microbial electrochemical sensor 202 may also comprise an output port 214 configured to receive the wastewater that has contacted the microbial electrochemical sensor 202.

Numerous configurations of microbial electrochemical sensor 202 are known in the art, and the present application is not limited to microbial electrochemical sensor 202 as shown in FIG. 2. For example, the relative location of the anode 204, the ion-exchange membrane 208, and the chamber 210 configured to receive the fuel and wastewater may vary so long as the fuel and wastewater contacting the microbial electrochemical sensor 202 allow an appropriate ion can be exchanged between the ion-exchange membrane 208 and the fuel and wastewater. Thus, in some embodiments, the anode 204 may be disposed between the ion-exchange membrane 208 and the chamber 210 configured to receive the fuel. Similarly, the cathode 206, the ion-exchange membrane 208, and the chamber 210 for receiving the fuel and wastewater may vary so long as the wastewater can contact the cathode 206 and an appropriate ion can be exchanged between the ion-exchange membrane 208 and the fuel. In some embodiments, the anode 204, the cathode 206, and the ion-exchange membrane 208 are hot-pressed together in the fuel cell. In some other embodiments, the microbial electrochemical sensor 202 may not comprise an ion-exchange membrane 208.

The anode 204 may be composed of an inert material that generally does not react with fuel that is oxidized. The anode 204 may be composed of, for example, but not limited to, one or more of carbon cloth, carbon paper, conductive plastic polymers, steel, reticulated vitreous carbon, activated carbon, glassy carbon, graphite, nickel foam, or any non-conductive material coated with a conductive paint. The cathode 206 can similarly be composed of inert materials such as those described above with regards to the anode 204.

The anode 204 may comprise microbes. In particular, the microbes may be in the form of a biofilm on the anode 204. Any suitable microbes for the purposes of the present application may be comprised in the anode 204. In particular, the microbes may be sensitive to the toxicants in the wastewater. The microbes may be comprised in the fuel which is provided to the chamber 210 through the input port 212.

The cathode 206 may comprise catalysts, microbes or a combination of both. According to a particular embodiment, the cathode 206 may be a biocathode. The catalysts may be any suitable catalyst for the purposes of the present invention. For example, the catalyst may include, but is not limited to, platinum, manganese, titanium, cobalt, indium, tungsten and a combination thereof. The microbes may be any suitable microbes for the purposes of the present invention. In particular, the microbes may be in the form of a biofilm on the cathode 206.

The system 200 may further comprise a first reservoir 216 which may be configured to contain the fuel and may be fluidly coupled to the input port 212. The fuel contained within the first reservoir 216 may be any suitable fuel for the purposes of the present application. The fuel may comprise a concentrated, semi-solidified, or solidified organic compound which may be biodegradable. In particular, the fuel may comprise at least one of the following, sodium acetate, glucose, sucrose, xylose, starch, cellulose, synthetic wastewater, domestic wastewater, food processing wastewater, and lignocellulosic biomass. For example, the first reservoir 216 comprising the fuel, which is a source of organics, may be fluidly coupled to the input port 212 via a conduit (e.g., one or more pipes). Thus, the fuel may be stored and delivered to the chamber 210 at an appropriate time.

The system may comprise second reservoir 218 which may be configured to contain the wastewater and may be fluidly coupled to the input port 212. The wastewater contained within the second reservoir 218 may be any of the wastewaters described above. For example, the second reservoir 218 may contain industrial effluent wastewater having oxidized heavy metals and may be fluidly coupled to the input port 212 via a conduit (e.g. one or more pipes). Thus, the wastewater may be stored and delivered to the chamber 210.

According to a particular embodiment, the second reservoir 218 may comprise two compartments, a clarifier compartment and a feed compartment. In the clarifier compartment, any particle having a size above a pre-determined size may settle in the clarifier compartment and the supernatant may flow into the feed compartment. The wastewater comprised as supernatant in the feed compartment is then fed into the microbial electrochemical sensor 202 through the input port 212. In particular, the clarifier compartment may be used when the wastewater is turbid and settling of big particles in the wastewater is necessary. When the wastewater is not turbid, the second reservoir 218 may comprise the feed compartment without a clarifier compartment.

The fuel from the first reservoir 216 may also be used to increase the concentration of organic materials comprised in wastewater being delivered to the microbial electrochemical sensor 202. In this way, the concentration of the organic materials in the wastewater entering the microbial electrochemical sensor 202 via the input port 212 is always maintained above a baseline. This may be necessary when the wastewater has low organic concentration.

There is also provided third reservoir 220 which may be configured to collect the wastewater which has contacted the microbial electrochemical sensor 202, and may be fluidly coupled to the output port 214.

The system 200 also comprises process controller 222. The process controller 222 may be an automated process controller. The process controller 222 may be configured to execute instructions for monitoring and analysing levels of toxicants in the wastewater. In some embodiments, the process controller 222 is configured to perform instructions for executing a method for monitoring and analysing wastewater or a sample suspected of containing heavy metals. The process controller 222 may be in communication with the various components in the system 200 to control monitoring and analysis of the wastewater.

The system 200 further comprises first flow control device 224 fluidly coupled to the input port 212 and configured to adjust the flow of the fuel through the input port 212 (e.g., flow from the first reservoir 216 to the input port 212). The process controller 222 may be in communication with the first flow control device 224 and may adjust the flow of fuel to the chamber 210. For example, the process controller 222 may receive measurement data indicating an amount of one or more heavy metals is above a pre-determined threshold. The process controller 222 may increase the flow of fuel from the first reservoir 216 to the input port 212 using the first flow control device 224, which may increase the rate that heavy metals are reduced in the microbial electrochemical sensor 202.

According to a particular embodiment, the first flow control device 224 may also be configured to adjust a flow of the fuel which comprises organics through the input port 212 (e.g., flow from the first reservoir 216 to the input port 212). The process controller 222 may be in communication with the first flow control device 224 and may adjust the flow of the fuel to the chamber 210. For example, the process controller 222 may receive measurement data indicating the level organic strength of the wastewater entering the input port 212 is below a pre-determined threshold. For the purposes of the present invention, wastewater of low organic strength is defined as wastewater in which the organic compounds are at a concentration which is not sufficient for baseline production of current, voltage, power or hydrogen (i.e. the current, voltage, power or hydrogen is below a pre-determined baseline level) by the microbial electrochemical sensor. The process controller 222 may increase the flow of the fuel comprising the organics from the first reservoir 216 via the input port 212 to the microbial electrochemical sensor 202, into which the wastewater is also being fed, using the first flow control device 224 which may provide baseline production of current, voltage, power or hydrogen. The first flow control device 224 may be, for example, but not limited to, a valve or a pump.

Second flow control device 226 may be fluidly coupled to the input port 212 and configured to adjust a flow of the wastewater through the input port 212 (e.g., flow from the second reservoir 218 to the input port 212). The process controller 222 may be in communication with the second flow control device 226 and may adjust the flow of the wastewater to the chamber 210. For example, the process controller 222 may receive measurement data indicating an amount of one or more heavy metals is above a pre-determined threshold. The process controller 222 may decrease the flow of the wastewater from the second reservoir 218 to the input port 212 using the second flow control device 226 which may increase exposure time of the wastewater to the chamber 210 to further lower the amount of one or more oxidized heavy metals in the wastewater. Another example of when the second flow control device 226 may be required to adjust the flow of the wastewater to the chamber 210 would be when the total number of microbial electrochemical sensors 202 in the system 200 is changed. In particular, when the number of microbial electrochemical sensors 202 is increased, the process controller 222 may increase the flow of the wastewater from the second reservoir 218 to the input port 212 using the second flow control device 226. The second flow control device 226 may be, for example, a valve or a pump.

Third flow control device 228 may be fluidly coupled between the third reservoir 220 and the second reservoir 218. The third flow control device 228 may be configured to adjust the flow of the wastewater from the third reservoir 220 to the second reservoir 218. In particular, the third flow control device 228 may be configured to provide a feed recirculation when there is no or low water in the second reservoir 218 as detected by a level sensor (not shown) comprised in the second reservoir 218 and which may be in communication with the process controller 222. As an example, the amount of wastewater sent from the third reservoir 220 to the second reservoir 218 may be controlled by the process controller 222 in communication with the third flow control device 228 and the level sensor. The lack of wastewater flow to the microbial electrochemical sensor 202 may lead to a drop in the generation of current/voltage/power/hydrogen because supply of organic compounds in the wastewater is disrupted. In addition to this, the level sensor comprised in the second reservoir 218 may also confirm that there is no wastewater in the second reservoir 218, and thus the level sensor may pass relay to the process controller 222. Subsequently, the process controller 222 may command the third flow control device 228 to allow water to flow from the third reservoir 220 to the second reservoir 218. When the amount of wastewater in the second reservoir 218 increases, the flow to the second reservoir 218 from the third reservoir 220 may be decreased or discontinued. In this way, the microbial electrochemical sensor 202 is continuously fed with water through the inlet port 212, irrespective of the availability of wastewater in the second reservoir 218. Accordingly, the fluctuations in current, voltage, power, or hydrogen produced by the microbial electrochemical sensor 202 are minimised, thereby preventing any false alarms of the presence of toxicants.

According to a particular embodiment, when there is no or low water in the second reservoir 218 as described above, the process controller 222, following relay passed from the level sensor comprised in the second reservoir 218, may adjust the flow of fuel from the first reservoir 216 to the input port 212 into which the wastewater from the third reservoir is also being fed, using the first flow control device 224 to ensure baseline production of current, voltage, power or hydrogen is maintained.

According to a particular embodiment, when the second reservoir 218 comprises a clarifier compartment and a feed compartment, the level sensor may be mounted in the clarifier compartment. However, when the second reservoir 218 does not comprise a clarifier compartment, then the level sensor may be mounted in the feed compartment.

Although FIG. 2 shows the third flow control device 228 fluidly coupled to the second reservoir 218, it would be understood by a person skilled in the art that the third flow control device 228 may be fluidly coupled to the input port 212 in a configuration that bypasses the second reservoir 218 (not shown). For example, a conduit may directly connect the third flow control device 228 and the input port 212. The third flow control device 228 may be, for example, a valve or a pump. Another alternative is that the system 200 may not comprise the third reservoir 220 and the wastewater which has contacted the microbial electrochemical sensor 202 may be channeled back to the second reservoir 218.

The system 200 may also comprise electrical sensor 230 electrically coupled to the anode 204 and the cathode 206. The electrical sensor 230 may be configured to measure at least one of a voltage, a current, power or amount of hydrogen produced between the anode 204 and the cathode 206. The electrical sensor 230 may be in communication with the process controller 222 and may provide measurement results for the electric current, voltage, power, or hydrogen produced between the anode 204 and the cathode 206. The process controller 222 may adjust certain operating conditions for the microbial electrochemical sensor 202 based on these measurements. For example, the process controller 222 may decrease a flow of wastewater to the chamber 210 using the second flow control device 226 when a current, voltage, power or hydrogen produced is below a pre-determined threshold. As another example, the process controller 222 may increase a flow of fuel to the chamber 210 using the first flow control device 224 when a current, voltage, power or hydrogen produced is below a pre-determined threshold. The electrical sensor 230 may be, for example, a voltmeter, an ammeter or potentiostat.

The process controller 222 may be coupled to or be in communication with an auto-sampler and a telecommunication system (both not shown). In particular, the auto-sampler may be configured to collect a sample from the wastewater when triggered by the process controller 222 to do so. Such immediate collection of a sample of wastewater may aid and enable the further analysis of the water quality parameters in a laboratory for the confirmation of the presence of toxicants in the wastewater.

Likewise, the telecommunication system may also be configured to generate a notification to a user of the system 200 when triggered by the process controller 222 to do so. The notification may be transmitted via any suitable means, such as, but not limited to, wireless signal, internet, a supervisory control and data acquisition (SCADA) system.

The process controller 222 may be optionally coupled to an input device, such as a keyboard, mouse, touchscreen, etc. The input device may allow a user to adjust various settings or variables for the process controller 222 that modifies how the system performs the method for processing the wastewater. The process controller 222 may include any type of a microprocessor, a microcontroller, a digital signal processor (DSP), or any combination thereof. The process controller 222 may also include system memory, such as any type of volatile memory (such as RAM), non-volatile memory (such as ROM, flash memory, etc.), or any combination thereof. The system memory may store instructions for performing any method disclosed herein.

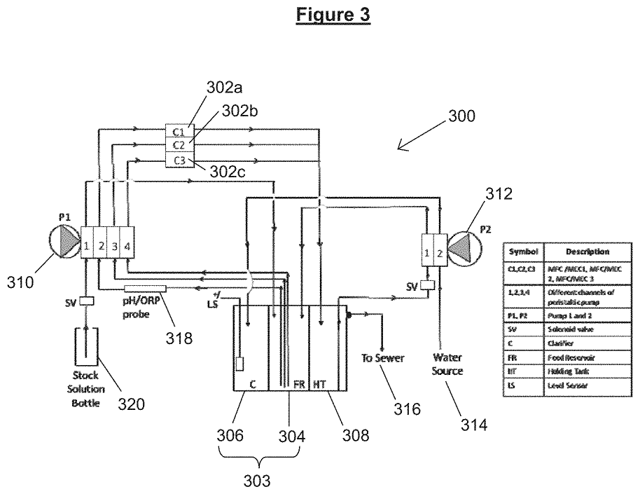

A method of using the system 200 will now be described with reference to FIG. 3. However, it would be clear to a person skilled in the art that FIG. 3 is only an example of a toxicant monitoring system and that many variations may be made to the system without departing from the present invention. FIG. 3 shows a system 300 based on the system 200 described above. In particular, there are three microbial electrochemical sensors, 302a, 302b and 302c. The microbial electrochemical sensors 302a, 302b and 302c may be as described in relation to microbial electrochemical sensor 202. The three microbial electrochemical sensors 302a, 302b and 303c may run simultaneously so that the presence of toxicants in the continuous feed water sample may be confirmed when more than 50% of the total number of microbial electrochemical sensors 302a, 302b and 302c detect the presence of toxicants.

As described above in relation to microbial electrochemical sensor 202, the anode surfaces of each of microbial electrochemical sensors 302a, 302b and 302c may be coated with a layer of biofilm. The biofilm may be grown on the surface of the anode. For example, a newly constructed microbial electrochemical sensor may be circulated continuously with a water source comprising natural mixed bacteria or any organic compound which contains pure culture or mixed bacteria for a period of time. The period of time may be any suitable period of time, such as 4 weeks. In particular, the water source comprising the bacteria or organic compound may be fed into the anode compartment from the bottom end of the microbial electrochemical sensor and discharged from the top of the microbial electrochemical sensor.

According to a particular embodiment, the microbial electrochemical sensors 302a, 302b and 302c may be microbial fuel cells. A microbial fuel cell is a fuel cell comprising an anode chamber and a cathode chamber, the anode and cathode being separated by an ion-exchange membrane. By way of an anaerobic process in the anode chamber, in which bacteria grows in the absence of oxygen, a biofilm that covers the anode is formed. In the anode chamber of a microbial fuel cell, organic matter may be metabolized by the biofilm comprising electrochemically active bacteria, thereby producing electrons which pass through an external circuit to the cathode where the reduction takes place. Hence, the current, voltage, or power produced is proportional to the utilization rate of the organic matter. When the biofilm is subjected to toxicants, the normal electron transport metabolism of the electrochemically active bacteria becomes inhibited, causing the current, voltage or power to decrease rapidly. Different types of toxic compounds of different concentrations would lead to different degree of reduction in the current, voltage or power generated. Therefore, the microbial fuel cell may be capable of quantitative, on-line and real-time sensing of toxicity in water. The current, voltage or power generated by a microbial fuel cell is dependent on the biochemical oxygen demand (BOD), oxidation-reduction potential (ORP), conductivity, pH and temperature of the water subjected to the sensor as these parameters affect the metabolism rate of the microorganisms.

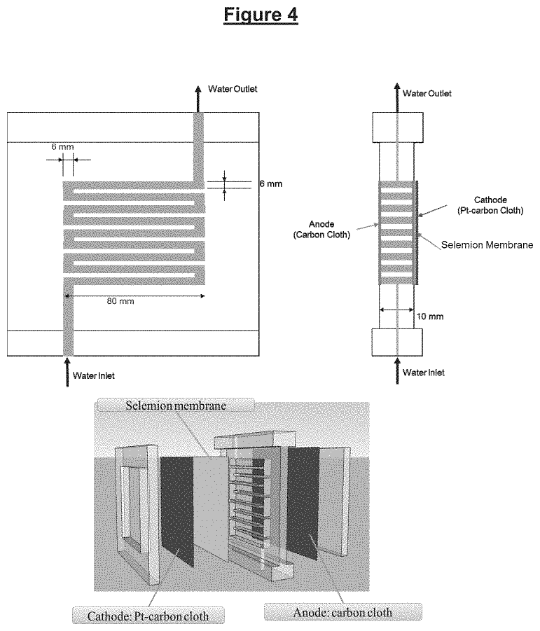

The microbial fuel cell (MFC) 302a, 302b and 302c as used in FIG. 3 is shown in FIG. 4. The microbial fuel cell may be a rectangular single-chambered flat plate air-cathode MFC which may be optimized according to the method as described in Cheng et al (Cheng et al, 2006, Environmental Science and Technology, 40(7):2426-2432). The MFC may have a flow channel within the anode chamber that allows a serpentine flow path to be created. The serpentine flow path may allow homogeneous microbial growth and may therefore increase sensitivity and reduce the response time of the monitoring system 300 to the toxicants present in the feed water. The water retention time may be as short as a few seconds by increasing the feed flow rate or reducing the size of the MFC. By way of an example, a rectangular air-cathode MFC may have an anode chamber of 8 cm long, 1 cm wide and 6 cm high. Channels in each MFC may result in the feed to follow a serpentine flow path of 1 cm wide and 0.6 cm deep, having a total working volume of 41.4 cm³. The anode and cathode surface areas of the MFC may be similar and may each be about 48 cm². Both the anode and the cathode may be made of carbon cloth (e.g., from supplier E-Tek, USA) and the cathode may be coated with platinum catalyst on one side (for example at a load of 0.5 mg cm⁻²).

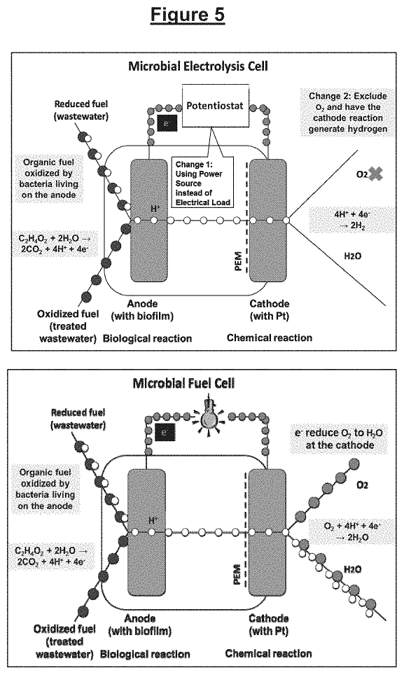

In an alternate embodiment, the microbial electrochemical sensors 302a, 302b and 302c may be microbial electrolysis cells. The differences between a microbial electrolysis cell (MEC) and a MFC are shown in FIG. 5. In particular, for a MEC, a constant potential difference must be applied to the anode and cathode. The presence of toxic compounds will be likewise indicated by a reduction of the current generated or monitoring the reduction of the production rate of hydrogen. The advantage of MEC is the elimination of the fluctuation of potential difference between the anode and cathode, which may be experienced by the MFC. Moreover, the sensitivity of the MFC against toxicity may be improved by the applied anode potential. This is shown in FIG. 6, which illustrates the improved sensitivity of MEC against Cu(II) when the anode potential is controlled at −0.1V vs. Ag/AgCl (3M KCl) reference electrode.

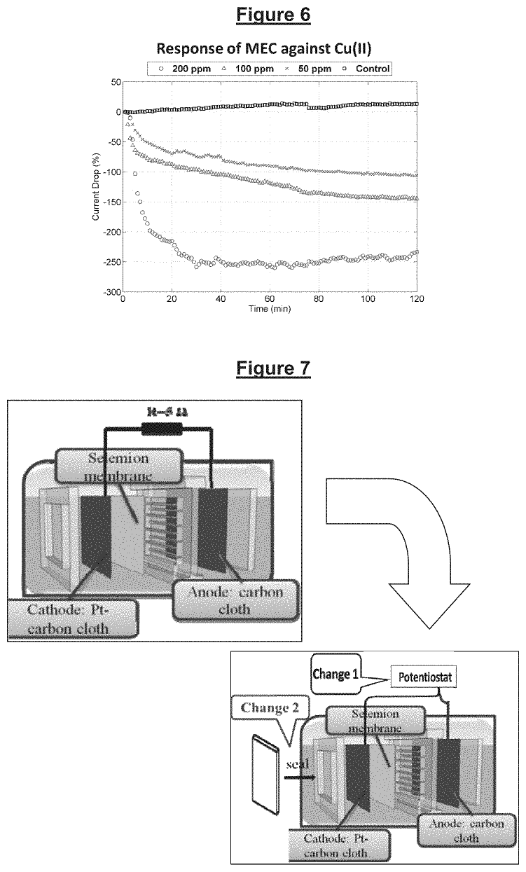

FIG. 7 shows how a MFC may be converted into a MEC by two changes: (1) using a potentiostat to fix the anode potential with reference to a reference electrode or directly applying a potential difference between the anode and cathode; and (2) sealing the cathode compartment to exclude oxygen. The hydrogen produced by the cathode reaction may be monitored, which may also serve as a signal to indicate the presence of toxicants in feed water.

Based on baseline current data of the microbial electrochemical sensor 302a, 302b, 302c, upper and lower limits of baseline current fluctuation (in percentage) may be defined (e.g. +10% to −10% fluctuation in the baseline current may be taken as the current fluctuation within the upper and lower limit). To detect and confirm the presence of toxic compounds in the continuous feed water sample being fed into the microbial electrochemical sensor 302a, 302b, 302c from a feed compartment 304 of a second reservoir 303, the algorithm embedded in process controller (not shown) will always compare the latest reading of current, voltage, power or hydrogen produced at time “t” with the reading of previous time step “t−n”, where “n” can be any value less than “t”.

The second reservoir 303 may be as described above in relation to second reservoir 218, and may comprise a clarifier compartment 306 and the feed compartment 304. As explained in relation to the second reservoir 218 above, in some embodiments, the second reservoir 303 may only comprise the feed compartment 304 without any clarifier compartment. In particular, it would be clear to a person skilled in the art that a clarifier compartment would only be required when the feed water entering the feed compartment is turbid, as explained above. As shown in FIG. 3, the feed from the feed compartment 304 may be controlled by pump 310 and its channels. The pump 310 may be any suitable pump such as a peristaltic pump.

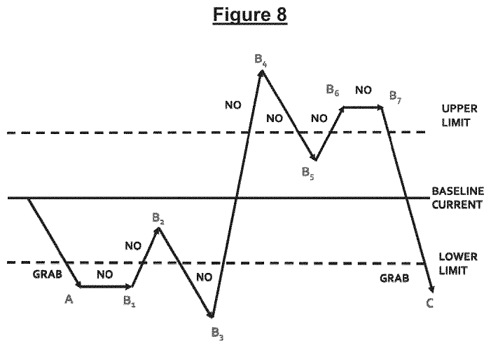

The working principle of the algorithm followed by the process controller is shown in FIG. 8. As illustrated in FIG. 8, in order to record any toxic event and trigger an auto-sampler to collect a sample, at least two out of the three microbial electrochemical sensors 302a, 302b, 302c must show the same trend. The basic steps of algorithm are as follows:

(a) If a current/voltage/power drop is detected to be more than the defined lower limit (e.g. −10%), the auto-sampler is triggered to grab a first sample immediately (point A of FIG. 8) and log-in the time and data of that event;
(b) If the current/voltage/power keeps decreasing or maintains itself beyond the defined lower limit, no sample shall be grabbed by the auto-sampler (point B₁);
(c) If the current/voltage/power fluctuates, yet does not cross the defined upper limit, no sample shall be grabbed by the auto-sampler (points B₂ and B₃);
(d) If the current/voltage/power crosses the upper limit, yet does not cross the defined lower limit, no sample shall be grabbed by the auto-sampler (points B₄ and B₅);
(e) If the current/voltage/power keeps on increasing or maintains itself beyond the defined upper limit, no sample shall be grabbed by the auto-sampler (point B₆); and
(f) If current crosses the defined upper limit and then drops beyond the defined lower limit, the auto-sampler shall be triggered by the process controller to grab a second sample immediately (point C) and log-in the time and data of that event.

It would be understood by a person skilled in the art that in order to carry out the algorithm exemplified above, the process controller would communicate with various other components of the system 200 as described in FIG. 2 but which may not necessarily be shown in the system 300 of FIG. 3.

Once the presence of toxic compounds has been confirmed by the process controller and the auto-sampler has grabbed the samples for further analysis, an alarm or message may be transmitted to a user of the system 200 or system 300 via a telecommunication system. The telecommunication system may comprise any suitable means, such as a wireless signal, internet, SCADA system or the like.

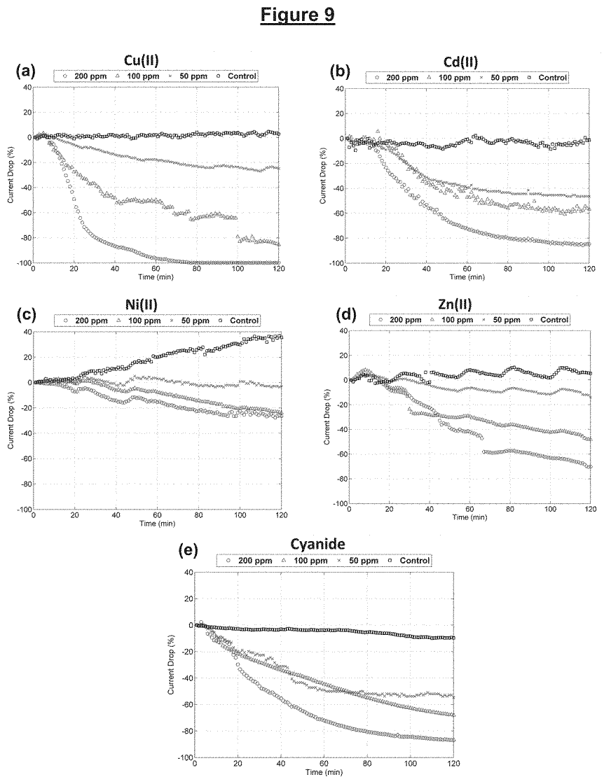

The system 200 and the system 300 may also estimate toxicant values, such as toxicant kind and toxicant concentration based on the current, voltage, power or hydrogen produced by the microbial electrochemical sensor. For example, system 300 exhibited rapid and proportional response to increasing toxicant concentration. The current drop-response time profile (Δl verses t) were obtained at different concentrations of Cu(II), Cd(II), Ni(II), Zn(II) and cyanide during a toxic event. Current drop pattern was found to vary with different metal kind as can be seen from FIGS. 9(a)-(e). From the obtained current drop profile at any defined response time of a wastewater sample containing heavy metal and cyanide, the toxicant may be estimated. In particular, the drop in produced current was more rapid and severe with increasing toxicant concentration.

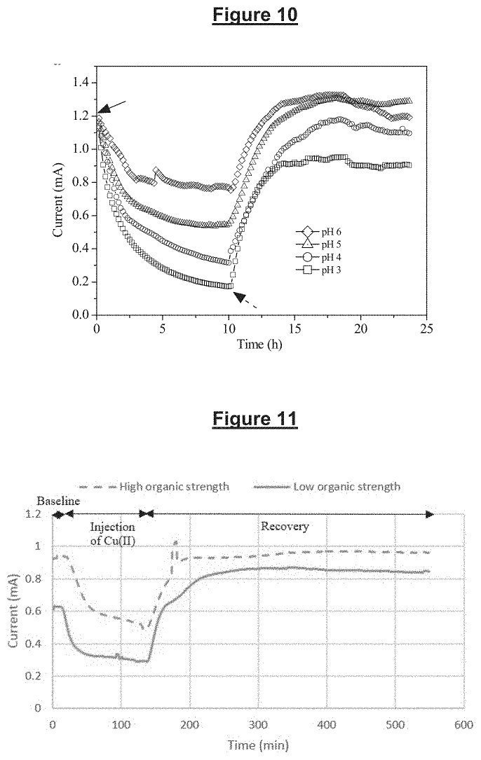

Other examples when there is a drop in production of current/voltage/power/hydrogen may be experienced is if there is a fluctuation in BOD, ORP or pH. Fluctuations in these would cause the baseline current/voltage/power/hydrogen to fluctuate. To exemplify this, there is provided FIG. 10 which shows that the system 300 was sensitive to monitor acidic toxicity of the wastewater being fed into the microbial electrochemical cells. In particular, the presence of acidic toxicity may lead to a fast drop of the current generation immediately. As can be seen from FIG. 10, the current dropped drastically during the first 4 hours and then slowed down subsequently. The decrease continued as long as the system 300 was exposed to the acidic pH and no stabilized minimum current was observed except for pH of 6. The extent of inhibition observed was found to correlate to different acidic pH, showing the existence of a dose-response relation.

In addition to the detection, collection of sample and alerting the user of the system 200 or system 300, the system 200 or system 300 may further comprise a diversion line (not shown) to divert a wastewater stream to a holding basin (not shown). In particular, the process controller, via appropriate algorithm, based on the current/voltage/power/hydrogen generation data may monitor the presence of toxicants in the wastewater stream and when toxicants are detected, the wastewater stream may be diverted away from further biological processes such as activated sludge in a wastewater treatment system. Such diversion may minimize harmful effects of the toxic wastewater to the biological processes. An example of an appropriate algorithm for such diversion may comprise the following:

(a) If a decrease in current as compared to the baseline level is detected, the incoming waste stream may be diverted to a temporary holding basin and the diverted waste stream may only be returned to the treatment aeration basin at a rate which does not damage sludge quality; and
(b) When the current returns to the baseline level, the incoming waste stream may be returned to the regular flow path (e.g., to the aeration basin).

In the event the wastewater in the second reservoir 218 of system 200 or the second reservoir 303 of system 300 has low organic strength, or if there is no wastewater in the reservoirs 218 and 303 as detected by the level sensor, the process controller 222 of system 200 and the process controller of system 300 will make adjustments in the functioning of the system 200 and system 300.

In the system 300, a pump 312 via channel 2 may pump water from a water source 314 to the first compartment 306 of the second reservoir 303. The pump 312 may be any suitable pump such as a peristaltic pump. Big particles present in the water may settle in the clarifier compartment 306 of the second reservoir 303 and the supernatant may overflow to the feed compartment 304 of the second reservoir 303. The holding tank 308 may be as described above in relation to third reservoir 220. The water in the holding tank 308 may overflow via an overflow line to sewer 316.

Pump 310 via channels 2, 3 and 4 may pump water from the feed compartment 304 of the second reservoir 303 into the microbial electrochemical sensors 302a, 302b, 302c. Microbial electrochemical sensors 302b and 302c may directly pump water from the feed compartment 304 of the second reservoir 303 while microbial electrochemical sensor 302a may pump water from the feed compartment 304 of the second reservoir 303 through a pH/ORP probe 318, thereby monitoring the pH and ORP values of the water.

Effluent water from the microbial electrochemical sensors 302a, 302b and 302c may be collected in the holding tank 308. The pump 310 via channel 1 may pump fuel comprising organics from a first reservoir 320 into the feed compartment 304 of the second reservoir where the fuel may be mixed with the feed water. The feed water may be that as directly obtained from a water source or that overflown from the clarifier compartment 306 of the second reservoir, if such a clarifier compartment is present. The mixed solution may then be fed to the microbial electrochemical sensors 302a, 302b and 302c. The first reservoir 320 may be as described above in relation to the first reservoir 216. FIG. 11 shows an example of a response from the microbial electrochemical sensors 302a, 302b and 302c to Cu′ as a toxicant present in feed water with high organic strength and low organic strength.

During a normal water flow situation, the pump 312 via channel 1 may be closed using a solenoid 3-way valve, or the like. However, when there is no water flow from the water source, the lack of wastewater flow from the water source such as from feed compartment 304 may lead to a drop in the generation of current/voltage/power/hydrogen by the microbial electrochemical sensors 302a, 302b, 302c because supply of organic compounds in the wastewater is disrupted. In addition, the level sensor mounted in the second reservoir 303 may pass relay to the process controller indicating a drop in the amount of wastewater.

Subsequently, the process controller may command the solenoid 3-way valve to open the channel 1 line of the pump 312, thereby allowing water to be pumped from the holding tank 308 to the feed compartment 304 of the second reservoir. This feed recirculation arrangement enables the system 300 to be fed at all times irrespective of the availability of wastewater in the water source, thereby minimizing the fluctuations in current, voltage, power or hydrogen produced by system 300.

Whilst the foregoing description has described exemplary embodiments, it will be understood by those skilled in the technology concerned that many variations may be made without departing from the present invention.

1. A toxicant monitoring system for continuously monitoring level of a toxicant in wastewater comprising:
a microbial electrochemical sensor;
an electrical sensor electrically coupled to the microbial electrochemical sensor, the electrical sensor configured to measure at least one measurement of voltage, current, power, or hydrogen produced, wherein a drop in the measurement correlates to the presence of a toxicant;
a process controller in communication with the electrical sensor, the process controller configured to execute instructions for monitoring the level of the toxicant in the wastewater, wherein the process controller is in further communication with an auto-sampler and a telecommunication system, the auto-sampler configured to collect a sample from the wastewater when triggered, and the telecommunication system configured to generate a notification when triggered;
an input port configured to provide fuel and wastewater to the microbial electrochemical sensor; and
an output port configured to receive the wastewater after the wastewater contacts the microbial electrochemical sensor.

2. The system according to claim 1, further comprising a first reservoir fluidly coupled to the input port, the first reservoir comprising the fuel.

3. The system according to claim 2, wherein the fuel comprises a source of organics.

4. The system according to claim 1, further comprising a second reservoir fluidly coupled to the input port, the second reservoir comprising the wastewater.

5. The system according to claim 1, further comprising a third reservoir fluidly coupled to the output port, the third reservoir comprising the wastewater which has contacted the microbial electrochemical sensor.

6. The system according to claim 2, further comprising a first flow control device fluidly coupled to the input port, the first flow control device configured to adjust flow of the fuel from the first reservoir to the input port.

7. The system according to claim 4, further comprising a second flow control device fluidly coupled to the input port, the second flow control device configured to adjust a flow of the wastewater from the second reservoir to the input port.

8. The system according to claim 5, wherein the third reservoir is fluidly coupled to the second reservoir via a third flow control device, the third flow control device configured to adjust a flow of the wastewater from the third reservoir to the second reservoir.

9. The system according to claim 8, wherein the process controller is further in communication with at least one of the first flow control device, the second flow control device and the third flow control device.

10. The system according to claim 1, wherein the microbial electrochemical sensor comprises an anode and a cathode, the anode comprising microbes.

11. The system according to claim 10, wherein the microbe comprised in the anode is in the form of a biofilm.

12. The system according to claim 1, wherein the microbial electrochemical sensor comprises at least a microbial fuel cell or microbial electrolysis cell.

13. The system according to claim 4, further comprising a level sensor comprised in the second reservoir and in communication with the process controller, the level sensor configured to send a signal to the process controller when there is no wastewater in the second reservoir.
